(19) United States
(12) Patent Application Publication
(10) Pub. No.: US 2022/0270757 A1
(43) Pub. Date: Aug. 25, 2022

(54) SYSTEMS AND METHODS TO COLLABORATE, TO TRAIN AN EXPERT SYSTEM AND TO PROVIDE AN EXPERT SYSTEM

(71) Applicant: INTELLIJOINT SURGICAL INC., KITCHENER (CA)

(72) Inventors: JOSEPH ARTHUR SCHIPPER, KITCHENER (CA); ANDRE NOVOMIR HLADIO, WATERLOO (US); JUSTIN AARON MICHAEL, KITCHENER (CA); RICHARD TYLER FANSON, STONEYCREEK (CA)

(21) Appl. No.: 17/632,029

(22) PCT Filed: Jul. 31, 2020
(86) PCT No.: PCT/CA2020/051053

§ 371 Date: Feb. 1, 2022

Related U.S. Application Data
(60) Provisional application No. 62/882,189, filed on Aug. 2, 2019.

(51) Int. Cl. G16H 50/20; G16H 50/30; G16H 10/60; G16H 20/00; G06N 3/08; G06N 20/20

(52) U.S. Cl. CPC G16H 50/20; G16H 50/30; G16H 10/60; G16H 20/00; G06N 3/084; G06N 20/20

(57) ABSTRACT

Systems, methods, etc. for collaboration enable receipt of an expert consultation providing a recommendation in a particular scenario. An example is a surgery consultation. Outcome monitoring may generate measures of outcomes to evaluate the recommendations. A predictive outcome expert system (e.g. a prognosis expert system for surgery) may be trained using the respective expert recommendations and the measures of outcomes to generate outcome predictions automatically. Such training data for the prognosis expert system may also comprise plan execution data (indicating how successfully the recommendation was actually achieved), post-procedure care data, etc. In an orthopedic context, planning data may be input to the prognosis expert system to evaluate a particular plan. The prognosis expert system may be utilized to optimize the plan responsive to the predicted outcomes. The prognosis expert system may be utilized to evaluate an expert's value based on the quality of the predicted outcome for a given recommendation.

### CROSS-REFERENCE

This application claims, in respect of the United States, a domestic benefit, and in respect of all other jurisdictions, Paris Convention priority, in and to U.S. Provisional Application 62/882,189, filed Aug. 2, 2019, the entire contents of which are incorporated herein by reference, where permissible.

### FIELD

This application relates to computer task collaboration, image processing such as using neural networks to define an expert system such as for medical image processing and for scenario (e.g. treatment) planning and more particularly to systems and methods to collaborate, to train an expert system and to provide an expert system.

### BACKGROUND

In many areas of practice, including orthopedic or other surgical practices, collaboration may provide better results and better outcomes. A patient's surgeon may desire to receive a consultation from an expert surgeon in the field to provide guidance to treat a particular indication. The consultation may include providing confirmation of the patient's surgeon's proposed plan or a providing a new plan to the requesting surgeon. Different experts may have specific or preferred methods or approaches to a same indication, providing different plans for the same set of facts.

Orthopedic and other surgical practices employ 2D and 3D image modalities such as x-rays, Computed Tomography (CT) and other imaging systems to prepare patient images. The image (which may be multiple images of a patient from different planes) are typically reviewed by a practitioner such as a surgeon to plan a surgery to treat an indication shown in the image. An example of an orthopedic practice is hip surgery such as total hip arthroplasty (THA) where a patient's hip joint is configured with implants. Various pre-operative patient measurements (data) relating to the patient's existing anatomy may be determined from the images. Various planning (e.g. proposed) data may be generated during a planning step by a surgeon for the patient to treat the indication. For example, a surgeon may generate desired, leg length, offset and other data as target for the surgery. Other patient data may also inform the planning such as demographic data (e.g. age, height, weight, BMI, other treated or untreated pathologies, etc.) or behavioral data (e.g. activities of daily life, profession, hobbies, activity level, etc.).

It is desired to provide to systems and methods to collaborate, to train an expert system and to provide an expert system.

### SUMMARY

Disclosed are various systems, methods and other aspects, including in an embodiment a system for collaboration to request and receive an expert consultation providing a recommendation in a particular scenario. An example consultation is a surgery consultation such as for an orthopedic surgery procedure and an example scenario is described by a patient's data. In an embodiment, data obtained from the collaboration system (e.g. the respective patient data and the recommendations (e.g. targets)) is utilized to train one or more expert systems to generate recommendations automatically. Respective expert systems may be trained using data from individual experts and/or a single expert system (e.g. for a same type of scenario/consultation) may be trained using data from all of the experts. Further, outcome monitoring my generate measures of outcomes with which to evaluate the expert recommendations. In an embodiment, a predictive outcome expert system (e.g. a prognosis expert system for surgery) may be trained using the respective expert recommendations and the measures of outcomes to generate outcome predictions automatically. Such training data for the prognosis expert system may also comprise plan execution data (indicating how successfully the recommendation was actually achieved), post-procedure care data, etc. In an orthopedic context, in an embodiment, planning data may be input to the prognosis expert system to evaluate a particular plan. In an embodiment, the prognosis expert system may be utilized to optimize the plan responsive to the predicted outcomes. In an embodiment, the prognosis expert system may be utilized to evaluate an expert's value based on the quality of the predicted outcome for a given recommendation.

### BRIEF DESCRIPTION OF THE DRAWINGS

FIG. 1 is a block diagram of a system according to an example herein.

FIG. 2 is an example of a patient image that is annotated with anatomical measurements and planning targets.

FIGS. 3, 4, 5, 6A, 6B, 6C, 7A, 7B, 7C, and 8 are screen shots of user interfaces for a collaboration system according to an example.

FIGS. 9A and 9B are flowcharts of operations for a collaboration system according to an example.

FIG. 10 is a block diagram showing input and output from expert models and how an output of one may be analyzed by another according to an example.

FIG. 11 is a flowchart of operations in accordance with an example.

FIG. 12 is a block diagram of a computing device, in accordance with an embodiment, useful as a component of the system of FIG. 1.

The present inventive concept is best described through certain embodiments thereof, which are described herein with reference to the accompanying drawings, wherein like reference numerals refer to like features throughout. It is to be understood that the term invention, when used herein, is intended to connote the inventive concept underlying the embodiments described below and not merely the embodiments themselves. It is to be understood further that the general inventive concept is not limited to the illustrative embodiments described below and the following descriptions should be read in such light.

### DETAILED DESCRIPTION

### System Overview

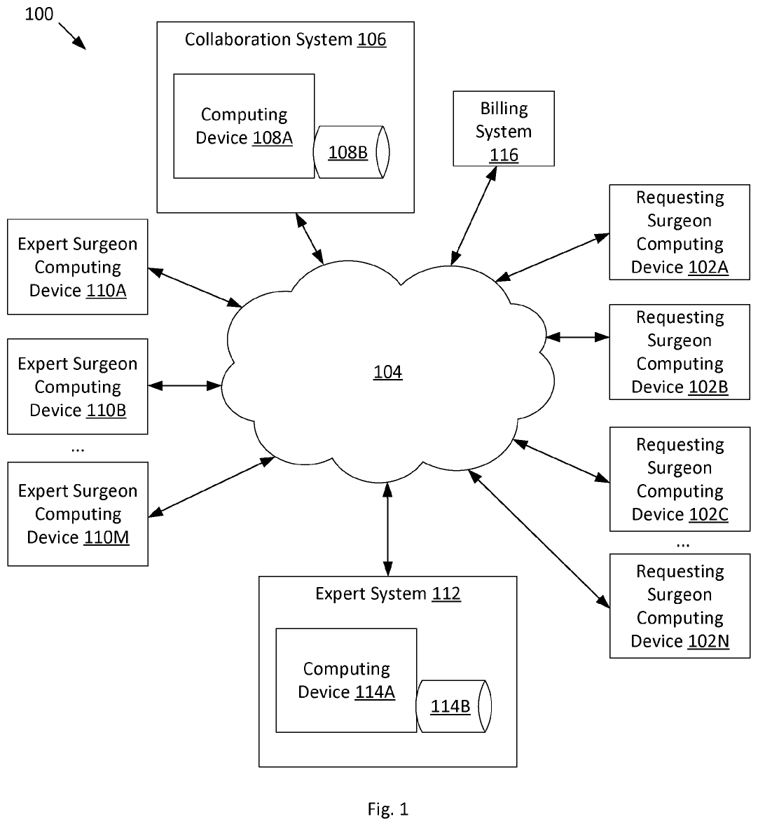

FIG. 1 is a block diagram of an example computer network system 100 in which a plurality of requesting surgeon computing devices 102A, 102B, 102C . . . 102N (collectively 102) are coupled via a network 104 to a collaboration system 106 comprising a computing device 108A and a database (data store) 108B. Similarly expert surgeon computing devices 110A, 110B, . . . 110M (collectively 110) are coupled via network 104 to collaboration system 106. N and M represent independent pluralities of such devices 102 and 104. There is also shown an expert system 112 comprising a computing device 114A and a database 114B coupled to network 104. Expert system 112 may communicate with collaboration system 106, requesting surgeon computing devices 102A, 102B, 102C . . . 102N, and expert surgeon computing devices 110A, 110B and 110C.

Each of requesting surgeon computing devices 102 may be configured to receive respective patient data such as from another source (not shown). Patient data may comprise patient image data. Patient data may be received from another system (not shown) via a communication or an intermediate storage device (not shown). Requesting surgeon computing devices 102 are typically operated by respective requesting surgeon users (also referenced as a regular surgeon or regular user herein, in contrast to an expert surgeon (or expert user) from whom consultations are requested). Via use of collaboration system 106, each of requesting surgeon computing devices 102 is configured to upload patient data and define respective planning data for a patient, and request a consultation (e.g. a review) of the patient data and planning data for the patient from one or more expert surgeons.

Collaboration system 106 is configured to receive the patient data and planning data and store such to database 108B. Collaboration system 106 may provide a web-based or other interface (e.g. a portal) to receive the patient data and to define planning data. Planning data may be received via an interface that permits a user of a requesting surgeon computing device to define the planning data as further described below. A request for consultation may comprise an indication of which one or more expert surgeons are to provide the consultation and a review deadline.

Collaboration system 106 also provides an interface (e.g. a web-based portal or otherwise) to each of the expert surgeon computing devices 110 (e.g. for use by their respective expert users) to communicate that a request for a consultation has been received and to provide the patient data and planning data from database 108B for review. For example, a request for a consultation may be communicated only to the respective expert surgeon computing device associated with the expert user who is to perform the review. The interface provides functionality to perform the review, for example to prepare the expert's planning data. The requesting surgeon's planning data may be maintained by collaboration system 106 such as for comparison. The interface may permit the expert to assign the review to another expert, for example because they do not have sufficient time to complete the review by the deadline. An assignment may be confirmed by the requesting surgeon.

Collaboration system 106 may be configured with respective user accounts for requesting surgeons and expert surgeons having respective login and password data as well as respective permissions, etc. within collaboration system 106.

The respective interfaces may comprise respective graphical user interfaces (GUIs) for example, comprising screens or other constructs. The interfaces may provide functionality to:

For a Requesting Surgeon:
manage the requesting surgeon's account (e.g. password, contact data, fees paid or payable)
request a new review (set deadline, name expert(s)) including up load data (e.g. and plan using such data)
receive a completed review including download data
monitor progress of a review (e.g. receive notices of change)
confirm or deny a request to transfer to another expert

For an Expert Surgeon:
manage the expert surgeon's account (e.g. password, contact data, fees received or receivable, fees paid or payable)
perform a review in reply to a request including up load data
assign review request to another expert

In an example, an expert surgeon may also be enabled to request a review and thus have permissions and abilities of a requesting surgeon.

Collaboration system 106 may employ workflow to assist with or guide a process to request and receive a review and to receive a review request and perform a review. Review milestone or status data may be maintained in database 108B and be reviewed such as through the GUIs. A screen or screens may be provided showing an expert surgeon's pending requests, work in progress, reviews completed, etc. A screen showing pending requests may have controls to invoke an interface to begin a review for a respective request. A review screen may have a control to save a review as a work in progress and a control to submit a completed review, etc. The review screen may be enabled with one or more controls to invoke an application such as to review an image or to download image data for local review, etc.

A submission of a completed review may trigger a message by collaboration system 106 to the requesting surgeon (e.g. within an interface of Collaboration system 106 or separately such as via an email, SMS, etc.) that a review is complete. The change in status may be viewable to a requesting surgeon through a screen showing the surgeon's requested reviews and their respective status.

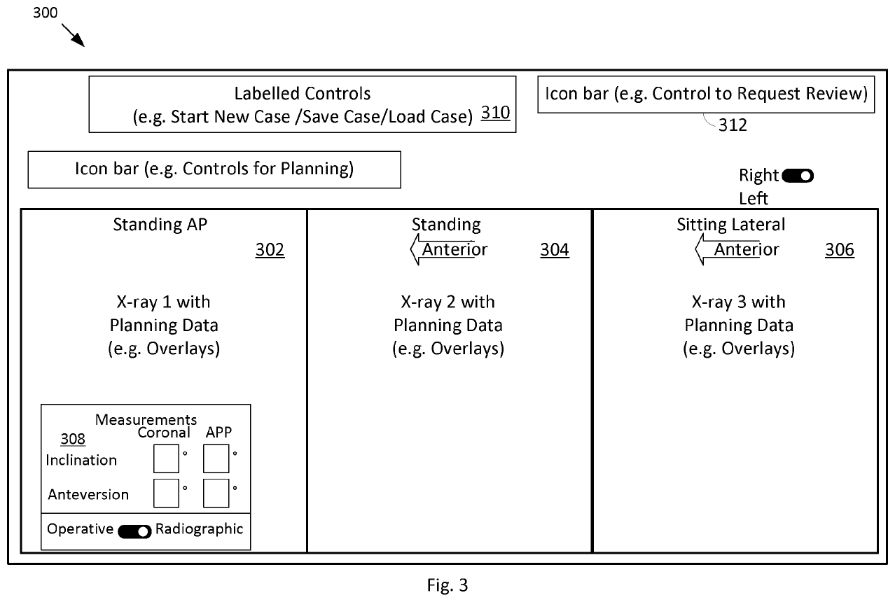

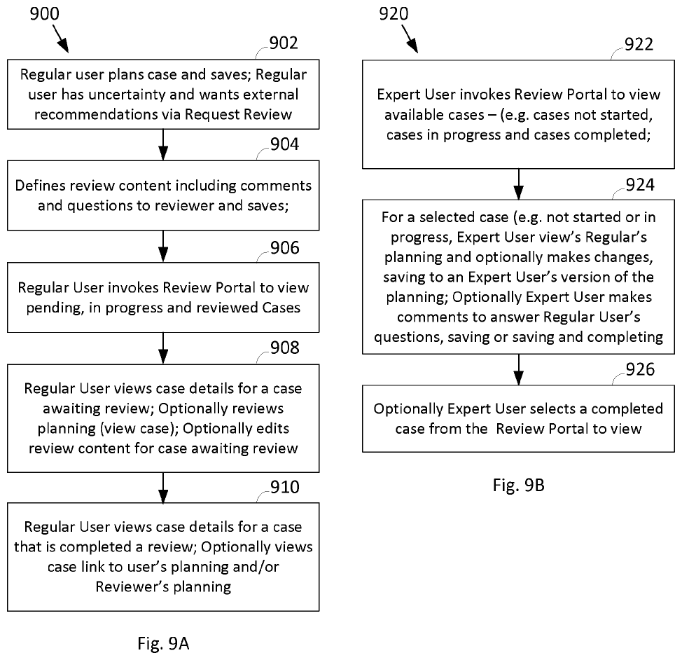

FIGS. 3, 4, 5, 6A, 6B, 6C, 7A, 7B, 7C, and 8, described further herein below, provide screen shots and FIGS. 9A and 9B a representative flow of operations of collaboration system 106 in accordance with an example.

### Fee Model

Collaboration system 106 may comprise or be linked with a billing system and/or payment system (e.g. billing system 116) such as via network 104 or a local network (not shown). Expert surgeons may be enabled to receive consultation fees through participation in collaboration system 106. Fees may be payable by requesting surgeons. Collaboration system 106, via billing system 116, may bill a fee per transaction (e.g. each request for consultation) to the requesting surgeons. A portion of the fee may be payable to the expert surgeon and a portion to operators of collaboration system 106. Annual or other fees may be billed to users (e.g. requesting surgeons and/or expert surgeons) depending on various fee models for use of collaboration system 106.

### Patient Data and Planning Data

A completed review typically comprises patient data and planning data. Any of the patient data and planning data may comprise patient image data (e.g. x-ray images or other images from other imaging modalities). Planning data may comprise initial planning data provided with the review. Planning data may comprise additional and/or replacement planning data. Additional or replacement planning data may comprise annotations to planning data provided by the requesting surgeon or new instances of planning data.

Patient image data may comprise or be associated with mark-up that indicates features of the patient anatomy shown in the image. Planning data may comprise targets for patient anatomy and/or implants for a patient as a portion of a procedure to treat the patient. A target herein may comprise an anatomical measurement, implant measurement or other measurement that is sought to be achieved through the procedure. For example, a target in a hip surgery may comprise a measure of anteversion (e.g. of X°). Planning data for a particular procedure may comprise a plurality of targets. Planning data may comprise additional data other than measurement data as further described below.

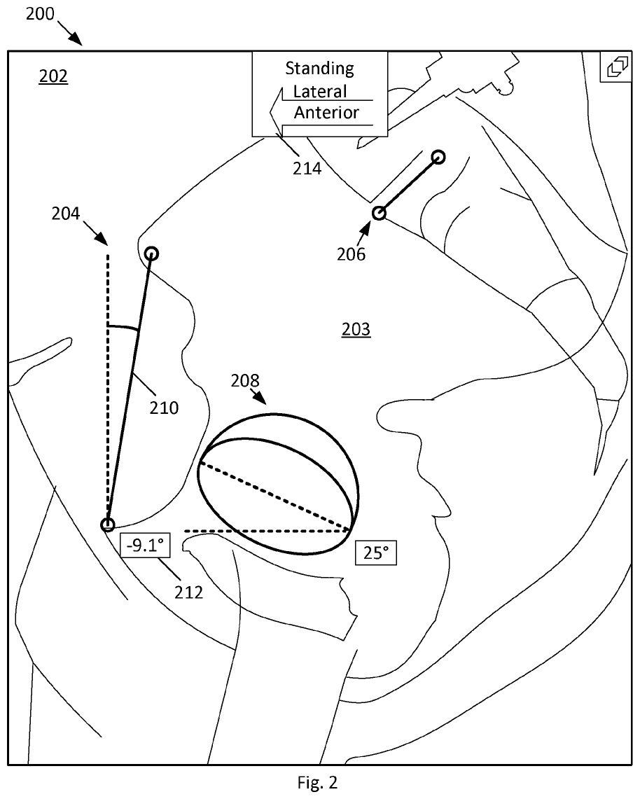

As shown in FIG. 2, there is shown an illustration of a portion of a screen shot 200 including an image 202 of a patient pelvis 203 in which certain anatomical landmarks or regions are indicated. Annotations (e.g. 204 and 206) are made to indicate the landmarks using controls of a user interface (not shown). One example of a region (specific portion of the anatomy) is an acetabulum. A region may be indicated by a bounding box or by coordinates of a shape (e.g. 208). A rectangle may be indicated by pixel coordinates of diagonal corners for example. One example of a landmark is the pubic symphysis. Two or more landmarks may be associated such as to define a line or plane (e.g. anterior pelvic plane 210). Multiple landmarks or planes may be combined to define a clinically relevant measure (e.g. as at annotation 204 and measure 212). Annotations and measurements may be made (and presented) in one or more different planes (coronal, sagittal, transverse/axial, etc.) or in different patient postures (supine, standing (as shown), seated, flexed seated, etc.) as shown in the patient images. The position shown in the image and a direction may be indicated (e.g. at labels 214). A planning interface may present two or more images simultaneously and update them all (e.g. see FIG. 3).

Patient and/or planning data may be indicated by positioning an overlay (shape e.g. 208) over a patient image. An interface may be provided to position the overlay. The overlay may be positioned in a plurality of planes. Handles (controls) may be provided to the image to move the shape in multiple degrees of freedom. Other input methods may also be provided to move the shape. For example, a text box may be provided to receive text input describing how to move the shape. A shape may be used to mark existing or baseline patient anatomy and capture (store same) and then adjust the shape to indicate planning data comprising a target. For example a hemisphere shape may be provided to mark a patient's native acetabulum showing existing patient anatomical measurements (e.g. native anteversion). The patient data may be captured as a ground truth. The shape may be manipulated to a desired position indicating target measurements or outcomes. The shape may represent an implant.

In addition to measurement data (e.g. cup position, version, etc.) as a target for planning data, planning data may include other data (which may be considered a target) such as implant data. Implant data may comprise a general class (e.g. acetabular cup, femur stem, etc.), a brand or type thereof (e.g. dual mobility acetabular cup), size, or any other characteristic of the implant.

Planning data may include a go/no go indication. That is, for some patients, a particular treatment may not be suggested and thus a no go outcome may be generated as the planning data by an expert in response to the patient data. No go planning data examples may have no further planning data (e.g. no implant measurement data) as such is not indicated.

Planning data may comprise alternative care and/or additional care data. For example, an alternative care data may comprise a direction to perform another treatment first before the primary treatment for which the review is sought. Alternative care data may be provided in a no go review. Additional care data may be an indication to perform an additional procedure whether before or after the primary treatment. For example, if may have been determined that spine surgery is also required. The additional care data may be that hip surgery is a “no go” until the spine surgery is completed. Thus alternative and/or additional care may be similarly considered.

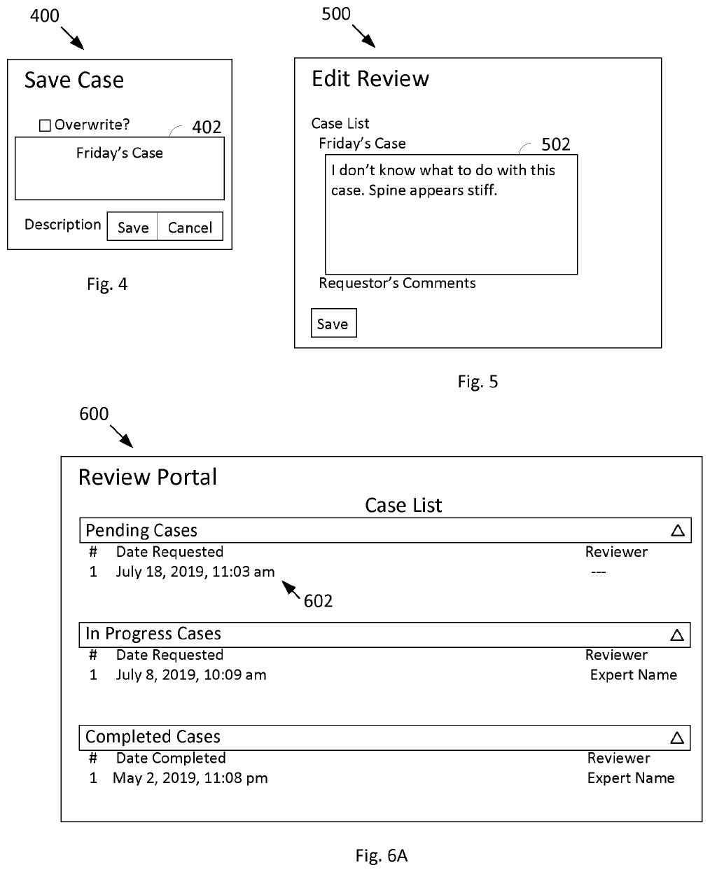

FIGS. 3, 4, 5, 6A, 6B, 6C, 7A, 7B, 7C, and 8 show screen shots (or portions thereof) and FIGS. 9A and 9B are flowcharts 900 and 920 showing a representative flow of operations of collaboration system 106 in accordance with an example. Operations may be invoked by and/or performed for a regular (requesting) surgeon (e.g. a regular user) and an expert surgeon (an expert user). FIG. 3 shows a representative planning interface 300 (from Intellijoint Surgical Inc. the applicant herein) showing patient images of patient anatomy. FIG. 3 shows three x-ray images (e.g. X-Ray 1 302, X-Ray 2 304 and X-Ray 3 306), of the same patient anatomy in three planes. The planning interface 300 permits annotation of the patient images (302-306) to indicate landmarks, make measurements and to place a shape (e.g. hemisphere) in a region of the patient anatomy and manipulate its position, which is updated in each of the different planes such as described and shown with reference to FIG. 2. Targets (e.g. inclination and anteversion 308, with reference to one or more planes) may be defined. Control(s) 310 are provided to save patient data and planning data in association with a case (e.g. for a patient) and a control 312 is provided to request a consultation (review) from an expert. FIG. 4 shows a save interface 400 for adding a description 402 (which may be a case name) to the case. When preparing the data for a review the requesting surgeon may make comments, ask questions of the reviewer and save the case. FIG. 5 shows an edit review interface 500 to edit a review, to add requestor comments 502. Save interface 400 and edit review interface 500 may be configured as overlays to interface 300. A billing system may be triggered in response to a request being initiated. With reference to FIG. 9A, steps 902 and 904 show operations as describe by way of example with reference to FIGS. 3 to 5.

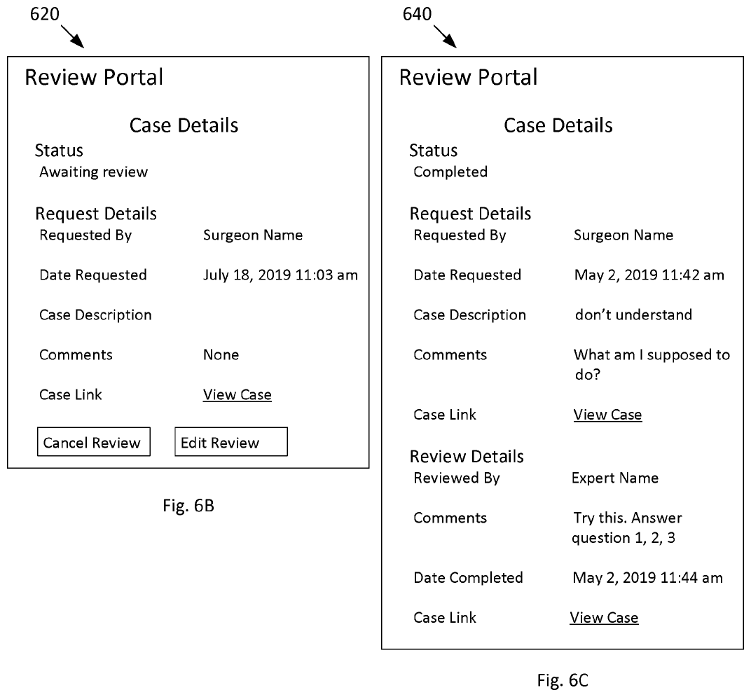

With reference to steps 906, 908 and 910, FIG. 6A shows a portal interface (e.g. Review Portal 600) to view pending, in progress and completed (e.g. expert reviewed) cases of the requesting user, as may be available. A requesting surgeon may see case details for and edit a request while pending (such as shown in FIG. 6B and interface 620) but not in progress or completed (once started the expert is allowed to finish). A case having a completed review is associated in the interface with a link (control) to invoke a review interface to present the case as the review. FIG. 6C shows a portion of a screen shot for a case that is completed. In the lists of case in Review Portal 600, an individual case (e.g. 602) may be selected and a control associated therewith may be invoked (e.g. a double click or tap) to invoke the associated interface (e.g. 620) to view the case details.

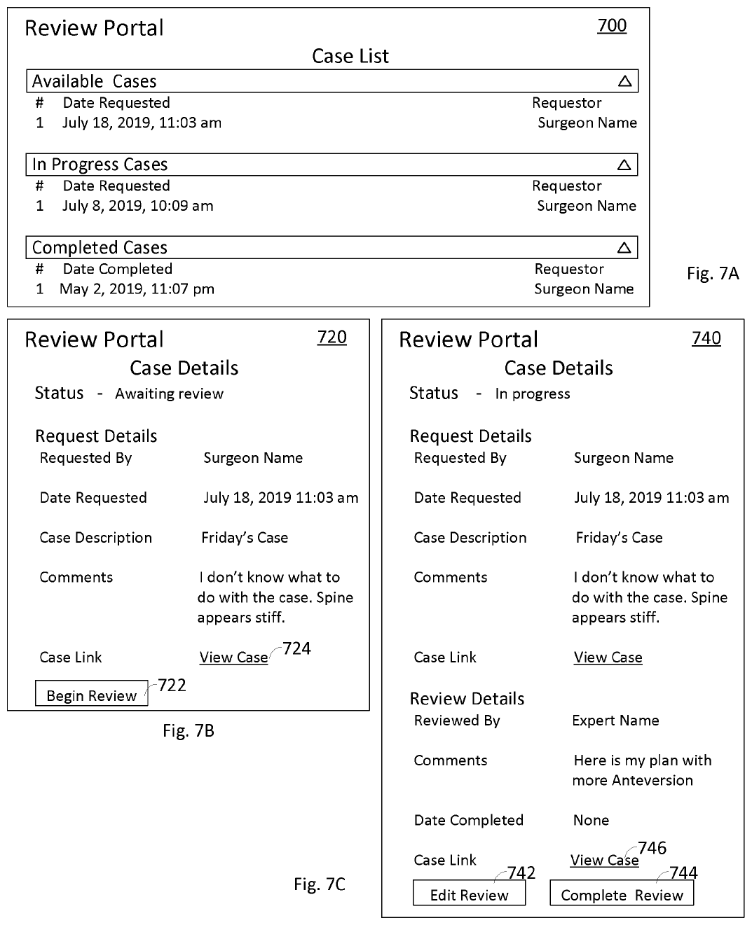

Turning to the flow of FIG. 9B and operations 920, a case is ready for reviewing. An expert may visit their review portal (e.g. interface 700 of FIG. 7A) to see available cases (an available case is one where a request for the expert to review has been made by the requesting surgeon), case in progress (where a review is started but incomplete) and cases that are completed. FIGS. 7A, 78 and 7C show screens displaying respective interfaces 700, 720 and 740 presenting a case list and respective case details for a case awaiting reviewed (720) and a case that is in progress (740).

As shown in the respective interfaces 720 and 740 of FIGS. 7B and 7C, the portal provides access to initiate a review (e.g. via control 722) for an available case and to continue a review (e.g. via control 742) or complete a review (e.g. via control 744) for an in progress case.

At step 924, an expert surgeon (via one of respective devices 110) reviews a case by beginning a new one from the available case list. The expert surgeon invokes the review interface by clicking on the case link 724 (to view but not overwrite the requesting surgeon's case). In the present example, the review interface is similar to the interface of FIG. 3 used by a requesting surgeon to define planning data. The start of a review interface may create a new review instance for saving within database 108B. Data from the request may be copied to the review instance. The expert surgeon works on the review instance data (not the data of the request itself). In this way there are separate records in database 108B and the original request is always available. “Original” here meaning the request data prepared by the requesting surgeon, which data may be changed by the requesting surgeon before the request is submitted or while the request is in a pending state only. The expert surgeon may save work in progress to database 108B. This work in progress may be picked up again (e.g. via case link 746) and further edited and/or completed. The status is also stored in the database such as for workflow use, tracking, statistics, billing, etc.

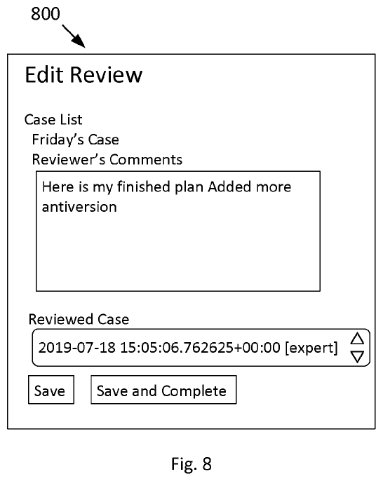

As shown in the screen shot of FIG. 8, in the edit review page (interface 800) the expert may select their reviewed case, and make comments to answer the questions of the requesting surgeon. The comments of the requesting surgeon may also be shown. Continuing with step 924, the completed case status is useful to associate the case with the expert surgeon's list of completed cases and to trigger a notice (e.g. email, not shown) to the requesting surgeon. The requesting surgeon's list of completed cases (via portal at 600) may also use this status. A billing system may also be triggered in response to the status. The requesting surgeon may view the review (new planning data, comments, etc. as well as the original request) (see FIG. 6C). Optionally, at 926, an expert user may review cases the user has completed (e.g. via interface 700). While the examples discuss and show a web-based approach to the collaboration system 106, a native application may be configured.

### Expert Planning Data Model

The patient data and planning data (target data) may be provided to train an expert system, that is, to train a model to produce planning data. In an example, planning procedures may include a surgeon annotating a patient image to determine measurements. These annotations and measurements may be ground truths for training data purposes. An expert system for image processing may be trained to process patient images to determine the measurements automatically, without need for a surgeon (or other user) to annotate the patient image.

Annotated images, particularly those having undergone a review by an expert, provide training data marked with ground truths and associated with target that are useful to train an expert system. The expert system, (such as expert system 112) may comprise a model such that the model may be trained to predict planning data targets from processing ground truth annotations. The model may be a linear or non-linear regression model, or a neural network.

The expert system, (such as expert system 112) may comprise a neural network for image processing such as a convolutional neural network (CNN). Annotated images are associated with the planning data targets such that a model of the network may be trained to predict planning data targets from processing the patient images. The annotations may provide ground truths for features in the images themselves. The targets provide ground truth predictions for the patient images. Loss functions may be defined to utilize the ground truths and train the model.

Additional inputs to the train the model may include items of patient data. Such items may include demographic information such as age, gender, ethnicity, other patient measurement data such as patient size related data (e.g. height, BMI), treated or untreated pathologies (e.g. hip, other joints), future treatment/proposed surgeries, treatment factor such as a surgical technique/approach, risk factors and comorbidities, including smoking, diabetes, scoliosis, down syndrome, desired Activities of Daily Living: stairs, skiing, biking, yoga, etc. By way of an example for a surgical approach in THA, the approach may be one of a posterior, an anterior or a direct lateral approach. By way of another example, a treatment factor may be an alignment philosophy for a knee procedure including one of a kinematic alignment, a measured resection and a gap balancing philosophy.

Thus, patient data herein may comprise identifying data. Identifying data may comprise patient name, address, case number, patient insurance information, etc. Patient data herein may comprise demographic data. Demographic data may comprise patient physical or related data comprising one or more of age; weight; height; BMI; gender; ethnicity; patient anatomy particulars comprising pre-operative anatomical measurements and existing implant data; medical history; treated or untreated pathologies; future treatment/proposed procedures; risk factors and comorbidities; and lifestyle/activities of daily living. Patient data may be in text or other form such as patient image data of patient anatomy.

Planning data herein may comprise medical procedure data such as a type of the medical procedure (e.g. a specific surgery), approach/philosophy to type, etc. Planning data herein may comprise at least one target for the medical procedure. A target may comprise an anatomical measurement, implant measurement or other measurement that is sought to be achieved through the procedure. Planning data herein may comprise a go/no go recommendation for the medical procedure.

Planning data herein may comprise an additional care recommendation— e.g. pre-procedure therapy/activity (e.g. reduce BMI/weight by X amount), post-procedure therapy/activity (e.g. physiotherapy, exercise, etc.) and/or an additional procedure (e.g. a second surgery).

Planning data herein may comprise an alternative care recommendation— e.g. for an alternative procedure (e.g. different surgery), therapy, etc.

Planning data may be in text or other form such as patient image data of patient anatomy.

In an example, a model may be trained to suggest a plan for THA by receiving an expert's base measures (e.g. horizontal reference, standing pelvic tilt, sitting pelvic tilt, sacral slope, pelvic incidence, lumbar lordosis), and receiving the expert's suggested output (e.g. cup inclination, cup anteversion, cup size, cup position) for a plurality of plans and using linear regression to predict the plan based on the inputs. In another example, the model may use loss functions to train a neural network to predict a plan.

Expert system 112 may comprise more than one model (or there may be more than one expert system). That is, a respective model may be generated from each respective expert surgeon's reviews. A plurality of such individual models may be generated. Each expert would thus generate at least a threshold number of reviews for model training, testing and validating purposes.

Expert system 112 may be configured to train new individual models through the use of transfer learning. Transfer learning is a machine learning technique in which a model trained for one learning tasking is used as the starting point for training a model for similar learning task. For example, a model for identifying jaguars in photographs may be developed by refining an existing model to identify house cats in photographs. Transfer learning may provide improved performance of a trained model where less training data is available for that model than would be required to fully train a new model. The expert system may use transfer learning to develop a model for an individual surgeon by refining a model developed for all other surgeons, reducing the number of reviews that the expert surgeon must provide before a suitable individual model may be generated.

In one example, an expert surgeon may be compensated (e.g. at rate $X/review) for each review generated until sufficient examples are generated to establish the model. Thereafter as the model is used to provide an expert review, the expert surgeon is compensated at $Y<$X/review.

In addition or alternatively, a more general model may be trained using reviews from all experts (or at least multiple experts).

Still further in addition or alternatively it may be that for some pathologies there are respective different approaches or surgical philosophies to treatment. Some experts may apply one approach/philosophy while others apply a second approach/philosophy, and so one. Respective models for each approach/philosophy may be generated from reviews from respective experts applying the respective approaches/philosophies. For example, some experts in THA may perform surgery in a direct anterior approach, while some may employ a posterior approach; models for each approach (or a single model that considers the approach as an input) may be generated. In another example, in TKA, kinematic alignment, measured resection and gap balancing may be input variables into the model, or there may be respective models for each philosophy. Alternatively, a review request may seek expert opinion on surgical approach and/or surgical philosophy. The review request may indicate this, and the expert review may provide a suggested surgical approach and/or philosophy an output. In a similar manner, the expert system may be configured to provide other options as either inputs or outputs (e.g. a requesting surgeon may seek an expert opinion on the implant system to use, or they may provide a specific implant system as a constraint on the expert plan).

Once trained, a requesting surgeon may be enabled to request an expert review (consultation) using one or more of the models. In one example, a request may be made for a specific expert. In one example a request may be made to a specific approach. A request may be made to more than one expert model. Results may be provided individually or to show a consensus, etc.

The system may be configured to perform training when a specific request is made by a requesting surgeon. In an example, a request may be made from two specific expert surgeons with a constraint to use an anterior approach THA. The example system may be configured to train a new model responsive to the request and its particulars if no such model already exists. The new model may be trained by using a unique subset of the training patient data available, selecting instances from the larger training data corpus that are relevant to the request. Such instances may comprise those that are substantially equivalent to the requesting patient data. If insufficient data exists to train a new model, a transfer learning technique may be employed.

In one example, a review may be requested and processed by a plurality of individual expert models (as a panel of experts) where the respective models are generated from training data of respective experts. The planning data output from the plurality of models may be compared and/or combined, for example to determine or select what a majority of the panel would do e.g. 5 out of 7 recommend cup angle of X°.

An interface may be configured to show the results from a plurality of experts, whether from different expert models or actual expert reviews of the same patient. For example an interface may be provided to present the patient image data and to overlay each recommendation (planning data targets). Such may be overlaid on a same image or on adjacent images. A shape for a particular expert may be illustrated in a specific colour. The interface may be configured to select or hide an expert's recommendation.

Collaboration system 106 and/or expert system 112 or another system may be configured to initiate a request, with patient data, to an expert surgeon (or more than one expert) to receive a review including planning data where the request is not generated by a requesting surgeon. The request may be generated by another user such as an administrator of collaboration system 106, expert system 112, etc. In this way additional ground truth data may be generated for training.

### Expert Prognosis Prediction Model

Expert system 112 may be configured to monitor patient outcomes following a procedure, for example, receiving post-procedure patient data, which may comprise image data to provide a further learning stream to train an expert prognosis prediction model.

Post-procedure patient data may include measurement data to verify (e.g. to provide comparative data) whether the target planning data was achieved. Was target X achieved and if not by how much? Post-procedure patient data may comprise a measure of patient outcome such as incidents of dislocation, revisions, readmissions, patient satisfaction, infections, bone fractures, etc. Post-procedure patient data may alternatively or additionally include information about the execution of the plan and any intentional deviations (e.g. if the surgeon intentionally implanted an acetabular cup at non-target angles to achieve a more stable bone fixation).

Post-procedure patient data may comprise post-procedure treatment data such a therapy data (e.g. course of therapy and completion data (e.g. “What therapy was prescribed and what was actually performed?” Did the patient adhere to therapy plan?)).

Expert system 112 may be configured to provide a predictive model trained from the planning data, patient data and post-procedure patient data whereby the predictive model is trained to predict a prognosis (e.g. a patient outcome). For example, the predictive model may take as input planning data, patient data and predict patient outcome data. The prediction may have confidence measures that are sensitive to measures of whether the planning data was achieved (e.g. using comparative data as described) and/or to measures of whether a patient adhered to therapy and/or patient data per se such as size data, activity data, etc. Analysis may be used to determine crucial parameters (i.e. those that are most sensitive for predicting particular patient outcomes). Analysis may determine that cup position is a sensitive parameter, or a relationship between two parameters such as body weight and another parameter. This information may be provided to computer systems (e.g. via a network connection) that are used to: select surgical tools and/or navigational systems; provide patients with patient-specific rehabilitation protocols and/or tailored educational content.

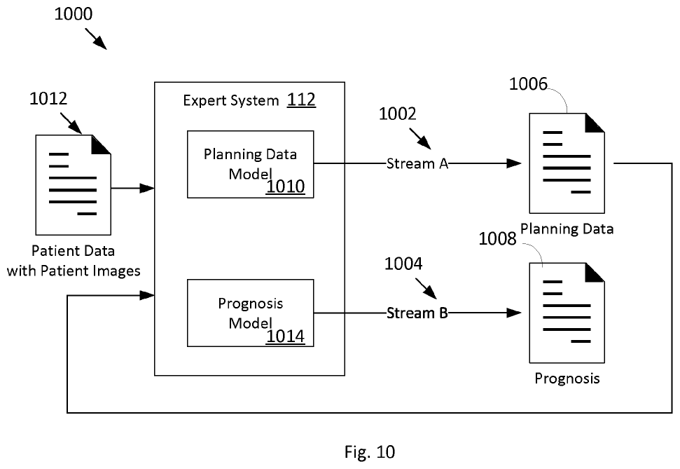

The predictive model may be used in various ways. FIG. 10 shows Stream A 1002 and Steam B 1004 comprising, respectively, planning data 1006 and prognosis data 1008 output from expert system 112. Expert system 112 in the present example has a planning data model 1010 for producing the planning data 1006 from patient data 1012 and a prognosis model 1014 for predicting a prognosis (in prognosis data 1014) from the planning data 1006 and the patient data 1012. In the present example, patient data comprises image data. Stream A 1002 output may be obtained by analyzing patient data as described. Stream B 1004 output may be obtained by analyzing the planning data 1006 such as is output from Stream A 1002 or otherwise obtained (such as from an expert surgeon or regular surgeon in the surgery example or other experts or non-experts in other contexts).

The prognosis model 1014 may be offered (made available electronically) in a similar fashion to the planning data model 1019. Planning data 1006 and any patient data 1012 used and input may be received from a requesting surgeon, who may not be an expert. The planning data 1006 and patient data 1012 may be processed and a prognosis provided as prognosis data 1008.

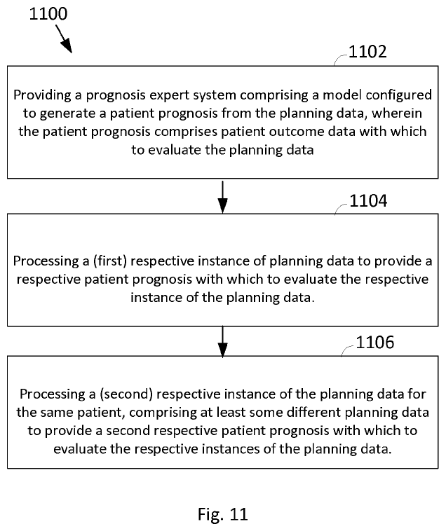

FIG. 11 shows a flowchart of operations 1100 for a method to evaluate planning data for a plan for a medical procedure. At step 1102, a prognosis expert system is provided comprising a model configured to generate a patient prognosis from the planning data. The patient prognosis comprises patient outcome data with which to evaluate the planning data. And, at step 1104, a respective instance of planning data is processed to provide a respective patient prognosis with which to evaluate the respective instance of the planning data.

The requesting surgeon may vary and resubmit the planning data 1006 and/or patient data 1012 with a view to optimizing the prognosis which in turn, optimizes the planning data and/or patient data. An iterative approach may be taken to improve the prognosis and thus optimize the planning data (and/or patient data). For example, a surgeon may vary a proposed target such as anteversion+/−1° to see if the prognosis is sensitive to this parameter. For example a surgeon may vary patient data such patient weight or BMI to see if such factors were improved the prognosis would also improve. The surgeon could propose a weight loss strategy.

Confidence measures may be provided. For example, the model may indicate that a particular prognosis may be achieved within a range of confidence e.g. 70-80%.

Sensitivities of the prognosis model may be provided to users. For example, through analysis it may be determined that a prognosis model is sensitive to patient size data. A change of patient weight of 10 kg may significantly improve a prognosis.

The prognosis model may be provided with planning data from two planning data models such as for different experts to generate respective prognosis for comparison.

An expert surgeon's consultative value could be evaluated. From an expert model trained on the experts' reviews, planning output may be fed through prognosis model. A measure of the prognosis could be determined. The expert could be ranked against specific peers or to the field of peers. The expert may lose their “expert” status due to poor prognosticative performance, and the expert system may remove that expert from the system.

The model may be used to steer away from a procedure. For example, the prognosis model may generate one or more prognosis that indicates the suggested treatment should not be performed, for example, because the one or more prognosis are all poor (e.g. the confidence intervals for any good prognosis are below a threshold). Thus at step 1106, in the example of operations 1100, a (second) respective instance of the planning data is processed for the same patient, comprising at least some different planning data to provide a second respective patient prognosis with which to evaluate the respective instances of the planning data.

A prognosis model interface (e.g. a portal) similar to FIG. 3 may be provided to receive planning data. A surgeon may request a prognosis once the planning data is generated. The prognosis may be provided via the interface. The requesting surgeon (in the present example referring to a surgeon requesting a prognosis and thus may or may not be an expert surgeon) may tweak various planning data and resubmit with a view to improving the prognosis.

The prognosis model interface may itself automatically generate multiple planning data requests each with slightly different planning data but without making large changes. Respective planning data items (e.g. individual items or elements of the planning data such as a specific target or two or more associated items, etc.) may have upper and lower change bounds and/or data perturbation rules defined, for example, to guide data perturbation by the interface for each request. Some items of planning data may have no changes. Thus a range of planning options are processed to produce a range of prognoses for the requesting surgeon.

In one example, two prognoses based on two plans may be provided, both with different potential complications (e.g. one plan may have a 10% chance of infection within 30 days, and another plan may have a 20% chance of patient dissatisfaction). Timing information may be included in the prognosis. The prognosis model interface may provide warnings to a user when certain types of complications are likely to occur within a certain timeframe (e.g. hip dislocation within 90 days of surgery). The type of complication and the timeframe may be based on insurance coverage of the particular patient. Some healthcare is payed for using bundled payments, where certain complications within a time window of the surgery must be covered out of pocket by the provider; hence, the aforementioned prognostic information may help in economic decision making.

In another example, surgical/treatment plans may include cost information. Cost information may be included in model training for given surgeries. For example, cost information may reside in a hospital information system database and/or derived from electronic medical records, and be linked to a specific case; this cost information may be accessed by the expert system. In another example, revenue information may be calculated for different prognoses and/or expert reviews. The revenue may be for the provider (i.e. hospital or surgery center) and/or for the surgeon. The revenue information may be calculated based on data available (e.g. through the Internet or a network connection) from payers (e.g. insurance companies). Both cost, revenue and risk information may be presented for each expert review and/or prognostic review, to help inform decision making by a requesting surgeon. The data and model training may show that a particular doctor (or maybe all doctors) generally factor cost in to their implant selection vs prognosis. It may be that there is a specific implant that costs $5000 more than an average comparable implant, but surgeons don't use it unless it decreases risk of dislocation by e.g. 20%. If it only decreases risk by 2% they wouldn't use it (even though it is technically better) because of the cost.

The two expert systems (or a single system comprising a plan model generating planning data and a prognosis model generating a patient prognosis for the plan) may be configured or otherwise utilized to optimize a prognosis, thus optimizing a plan while minimizing a plan's cost. Here the plan's cost may be short term costs such as those related to the procedure as well as longer term costs such as post-procedure costs.

Surgeons may have planning tendencies. Some may more rigorously follow particular approaches or styles. Evaluating outcomes may enable the identification of particular tendencies, approaches/styles that yield more desired outcomes (e.g. at least those outcomes toward the higher quality outcome— a maximization). Or such evaluation may enable the identification of specific surgeons providing consistently higher quality outcomes. As such the expert system or systems may be configured to or used to determine which surgeon or type of surgeon yields higher quality outcomes such as for a specific medical procedure.

It may be that an insufficient set of training data is available to train a robust prognosis model for wide variations in patient and planning data. Such may be the case because as prognosis monitoring may take many years to build sufficient data examples. A prognosis interface may be configured to constrain prognosis requests, filtering out those that may not produce good output for example because there is insufficient comparable training data. A request for a prognosis may be converted and sent as a request for planning data from an actual expert or from one or more expert planning data models.

The expert system may be configured to detect when a patient is significantly different from the patients used to train the model. In an example, a patient may be 22 years of age, but the model was only trained on a substantial number of individuals with ages from 45 to 80 years of age. The prognosis system may alter the age to be 45, and provided a notice for the surgeon of the behaviour, in addition to the prognosis.

The prognosis expert system may be configured with any one of a number of machine learning models to generate a prognosis based on patient and plan data. In one example, a linear regression model may be used to generate a single prognosis score (e.g. binary, integer, etc.). In another example, a back-propagation neural network may be trained to generate a score for each of a plurality of desirable or undesirable outcomes (e.g. change of dislocating, expectation of perceived stability).

The prognosis system may be configured to use multiple machine learning models sequentially to predict the prognosis data. In an example, a linear regression model may be used to predict a binary prognosis score for a specific outcome (e.g. dislocation), and a neural network model used to predict the timeline associated with the specific outcome.

Though described here in relation to orthopedic surgery other specialties and practices may be similarly modelled from collaborative data and from prognosis data. Others may include non-orthopedic surgery, dentistry, etc.

In accordance with respective embodiments there are provided systems (e.g. computing devices), methods, computer program products and other aspects.

In an embodiment, there is provided a method to evaluate planning data for a plan for a medical procedure comprising: providing a prognosis expert system comprising a model configured to generate a patient prognosis from the planning data, wherein the patient prognosis comprises patient outcome data with which to evaluate the planning data; and processing a respective instance of planning data to provide a respective patient prognosis with which to evaluate the respective instance of the planning data.

The method may comprise processing two or more respective instances of the planning data to provide respective patient prognoses with which to evaluate the respective instances of the planning data and optimize the planning data. The respective instances of the planning data comprise data items that are different.

In the method the respective instances of the planning data may be automatically generated using data perturbations generated in response to at least one of upper and lower change bounds and data perturbation rules defined to guide data perturbation.

In the method, the model may be trained using: respective planning data from respective expert consultations in respect of respective patients; and respective post-procedure patient data for the respective patients.

In the method, the post-procedure patient data may comprise any of: patient outcome data comprising patient satisfaction, incidents of dislocation, revisions, readmissions, infections, bone fractures; patient measurement data; plan execution data; and post-procedure treatment data.

In the method, the planning data to be evaluated may be generated by an consultation expert system configured to process patient data for a patient to provide an expert consultation comprising the planning data.

In the method, either or both of the prognosis expert system and the consultation expert system may be trained using cost data for plans defined by respective planning data and wherein the prognosis expert system and the consultation expert system are used or configured to generate the planning data to optimize the patient prognosis while minimizing a plan cost.

In the method, a quality of the patient prognosis may be useful to evaluate an expert associated with the expert consultation.

In the method, the patient data may comprise: demographic data comprising patient physical or related data comprising one or more of age; weight; height; BMI; gender; ethnicity; patient anatomy particulars comprising pre-operative anatomical measurements and existing implant data; medical history; treated or untreated pathologies; future treatment/proposed procedures; risk factors and comorbidities; and lifestyle/activities of daily living.

In the method, the planning data may comprise medical procedure data comprising one or more of a type of the medical procedure and an approach/philosophy to type; a target for the medical procedure selected from an anatomical measurement, an implant measurement and another measurement that is sought to be achieved through the procedure; an additional care recommendation comprising one or more of a pre-procedure therapy/activity, a post-procedure therapy/activity and an additional procedure; and an alternative care recommendation comprising an alternative procedure and/or an alternative therapy. In the method, the medical procedure is an orthopedic surgery procedure.

In an embodiment there is provided, a method to collaborate comprising: defining a request for an expert consultation to be provided by an expert; communicating the request to the expert via a collaboration service; and receiving the expert consultation.

In the method, defining the request may comprises providing patient data and optionally planning data to the collaboration service for communication to the expert.

In the method, defining the request may comprise identifying the expert to perform the expert consultation.

The method may comprise monitoring a progress of the expert consultation via the collaboration service.

The method may comprise providing an on-line portal for receiving the collaboration service, the on-line portal configured to provide an interface to define the request, to monitor the expert consultation and to receive the expert consultation.

In an embodiment there is provided a method to collaborate comprising: receiving from a requestor a request for expert consultation to be performed by an expert; communicating the request to the expert; and receiving the expert consultation for communicating to the requestor thereby to provide a collaboration service.

In the method, receiving the request may comprise receiving patient data and optionally planning data for communication to the expert.

The method may comprise providing a request interface to the requestor to define the patient data and optionally the planning data.

In the method, the request may comprise an identification of the expert to perform the expert consultation.

The method may comprise providing a monitoring interface to the requestor to monitor progress of the expert consultation.

The method may comprise providing an expert consultation delivery interface to communicate the expert consultation to the requestor.

The method may comprise providing a billing service to bill the requestor in response to a status of the expert consultation.

The method may comprise providing a performance interface to the expert to define the expert consultation, the interface configured to save a partially completed expert consultation as a work in progress. The method may comprise providing a work in progress interface for the expert to update or complete the partially completed expert consultation.

The method may comprise updating a respective status of requests from respective requestors and expert consultations from respective experts to a database and providing interfaces to requestors and experts, responsive to the status, to monitor progress.

In an embodiment there is provided a method comprising: receiving a request, from a requestor via a collaboration service, to prepare an expert consultation; preparing the expert consultation; and providing the expert consultation to the collaboration service for communicating to the requestor.

In the method, the request may comprises patient data and optionally planning data. In the method, preparing the expert consultation may comprise defining expert planning data. The method of any one of claims 30 to 32 comprising saving a partially completed expert consultation as a work in progress. The method of any one of claims 30 to 33 comprising receiving payment information from the collaboration service responsive to performing the expert consultation.

In an embodiment there is provided a method to train an expert system comprising: providing a collaboration service configured to receive respective expert consultations from an expert in response to requests for the expert consultations from requestors in respect of respective scenarios; defining instances of training data from instances of the respective expert consultations and the respective scenarios; and training an expert system using the instances of training data. In the method, the expert consultation may comprise a surgical consultation. In the method, the collaboration service may be further configured to receive a request from a respective requestor and communicate the request to the expert. In the method, the expert system may comprise a model to generate planning data for a medical procedure and the expert consultation may comprises, in respect of a particular patient, planning data generated for the patient's medical procedure and the respective scenario for the particular patient is defined by patient data. The method may further comprise: receiving post-procedure patient data for respective patients after the performance of respective medical procedures; and training a second expert system comprising a model to predict a patient prognosis using respective expert consultations for the respective medical procedures and the post-procedure patient data for the respective patients. In the method, the post-procedure patient data for a respective patient comprises any of: patient outcome data comprising patient satisfaction, incidents of dislocation, revisions, readmissions, infections, bone fractures; patient measurement data; plan execution data; and post-procedure treatment data.

In the method, the model of the second expert system may comprise one or more of: a linear regression model to generate a single binary prognosis score; and a back-propagation neural network to generate a score for each of a plurality of outcomes.

In the method, the second expert system may comprise multiple machine learning models employed sequentially to predict the patient prognosis.

In the method, the second expert system may comprise a linear regression model to predict a binary prognosis for a specific outcome and a neural network model to predict a timeline associated with the specific outcome.

In an embodiment, there is provided a method to provide an expert consultation in respect of a plan for a medical procedure proposed for a patient, the method comprising: receiving a request from a requestor for the expert consultation, the request comprising patient data in respect of the patient; processing the request using an expert system configured to generate the expert consultation from the patient data, wherein the expert consultation comprises planning data to execute the medical procedure; and communicating the expert consultation to the requestor.

In the method, the expert consultation may comprise a go/no go recommendation in respect of the performance of the medical procedure. In the method, the medical procedure may comprise an orthopedic surgery procedure.

In the method, the patient data may comprise at least one patient image and the expert system comprises a deep neural network to process the at least one patient image and generate at least one target as a portion of the planning data, wherein the at least one target comprises an anatomical measurement, implant measurement or other measurement that is sought to be achieved through the medical procedure.

In accordance with an embodiment, there is provided a computer implemented method to provide a patient prognosis in respect of a plan for a medical procedure proposed for a patient, the method comprising: receiving a request from a requestor for the patient prognosis, the request comprising planning data for the plan; processing the request using an expert system comprising a model configured to generate the patient prognosis from the planning data, wherein the patient prognosis comprises patient outcome data with which to evaluate the planning data; and communicating the patient prognosis to the requestor.

In any of the various methods herein, and as is applicable: the expert consultation may comprise a surgical consultation; the medical procedure may comprise an orthopedic procedure; the patient data may comprise demographic data comprising patient physical or related data comprising one or more of age; weight; height; BMI; gender; ethnicity; patient anatomy particulars comprising pre-operative anatomical measurements and existing implant data; medical history; treated or untreated pathologies; future treatment/proposed procedures; risk factors and comorbidities; and lifestyle/activities of daily living; the planning data may comprises medical procedure data comprising one or more of a type of the medical procedure and an approach/philosophy to type; a target for the medical procedure selected from an anatomical measurement, an implant measurement and another measurement that is sought to be achieved through the procedure; an additional care recommendation comprising one or more of a pre-procedure therapy/activity, a post-procedure therapy/activity and an additional procedure; and an alternative care recommendation comprising an alternative procedure and/or an alternative therapy; the patient data and planning data may each comprise one or more of text data and patient image data of patient anatomy; the model of the expert system may comprise one or more of: a linear regression model to generate a single binary prognosis score; and a back-propagation neural network to generate a score for each of a plurality of outcomes; the expert system (or model) may comprise multiple machine learning models employed sequentially to predict the patient prognosis; and the expert system (or model) may comprise a linear regression model to predict a binary prognosis for a specific outcome and a neural network model to predict a timeline associated with the specific outcome.

For any of the various methods herein, there is provided a computing device comprising a processor and a storage device coupled thereto, the storage device storing instructions which, when executed by the processor, configure the computing device to perform the method. Corresponding computer program products are also provided.

Any of the expert computing devices 110 and requesting surgeon computing devices 102 may comprise a computing device that is typically used for planning a surgery such as a laptop, desk top, workstation or other computing device. The device may be mobile/small form factor like a tablet or a laptop. A smartphone may be employed such as for at least some functions such as review status monitoring, account management, fees, etc. but is not typically used to review medical images.

Devices of collaboration system 106 and expert system 112 may comprise one or more hardware servers. It is understood that FIG. 1 is simplified and that various network components, etc. are not shown.

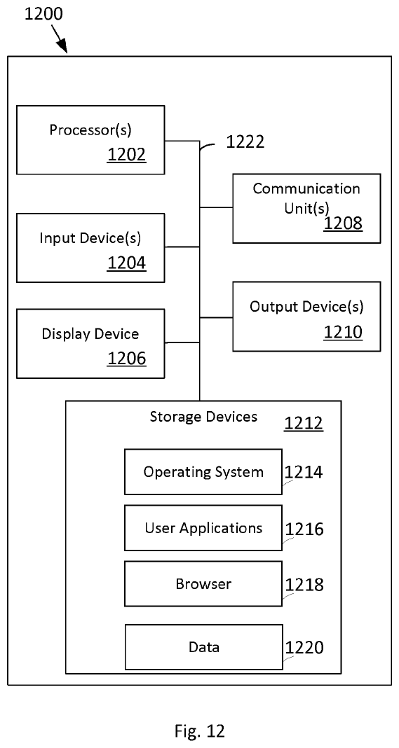

FIG. 12 is a block diagram showing a representative computing device 1200 such as may be configured as one of devices 102 or 110. It will be appreciated that the representative computing device 1200 of FIG. 12 may be also configured, with adaptations as may be required, as a server device such as one of devices 108A and 114A or of billing system 116.

The representative computing device 1200 comprises one or more processors 1202, one or more input devices 1204, a display device (which may be a gesture-based I/O device) 1206, one or more communication units 1208 and one or more output devices 1210. It will be appreciated that the display device 1206 is an output device and a gesture-based I/O device is both an input and output device. Some components may be optional or additional.

The representative computing device 1200 also comprises one or more storage devices 1212 storing one or more modules (e.g. 1214, 1216, and 1218) and/or data 1220. In a user configuration such as in devices 102 or 110, storage devices 1212 may store an operating system 1214, user applications 1216 such as for personal information management and/or for communication such as an email client, SMS client, phone client, contacts, calendar etc., an Internet browser 1218, etc. Other applications may be stored. In some examples, collaborative application 106 is a native application based application with a user application component stored in the storage device of the user device. In other examples collaborative application 106 is offered as a browser based application which may be accessed via an Internet browser 1218.

Features of the expert system such as the expert models may be provided in a cloud like application where data is communicated to the cloud based expert model(s) and output results are returned. In another paradigm the expert model(s) may be hosted by the user device such as devices 102 or 110 having suitable storage and processing resources. Thus a storage device may store an expert system model such as a deep neural network model (e.g. from a CNN), etc.

Communication channels, such as a bus 1222, may couple each of the components of the representative computing device for inter-component communications, whether communicatively, physically and/or operatively. In some examples, communication channels may include a system bus, a network connection, an inter-process communication data structure, or any other method for communicating data.

One or more of the communication units 1208 may communicate with external devices (e.g. a server of FIG. 1 or another computing device) such as for the purposes as described and/or for other purposes (e.g. printing) such as via the communications network of FIG. 1 by transmitting and/or receiving network signals on the one or more networks. The communication units 1208 may include various antennae and/or network interface cards, chips (e.g. Global Positioning Satellite (GPS)), etc. for wireless and/or wired communications.

The one or more storage devices 1212 may take different forms and/or configurations, for example, as short-term memory or long-term memory. Storage devices may be configured for short-term storage of information as volatile memory, which does not retain stored contents when power is removed. Volatile memory examples include random access memory (RAM), dynamic random access memory (DRAM), static random access memory (SRAM), etc. Storage devices 1212, in some examples, also include one or more computer-readable storage media, for example, to store larger amounts of information than volatile memory and/or to store such information for long term, retaining information when power is removed. Non-volatile memory examples include magnetic hard discs, optical discs, floppy discs, flash memories, or forms of electrically programmable memory (EPROM) or electrically erasable and programmable (EEPROM) memory.

Though not shown, a computing device may be configured as a training environment to train an expert system for example using a network model along with appropriate training and/or testing data.

The expert model may be adapted to a light architecture for a computing device that is a mobile device (e.g. a smartphone or tablet) having fewer processing resources than a “larger” device such as a laptop, desktop, workstation, server or other comparable generation computing device.

The one or more processors 1202 may implement functionality and/or execute instructions within a computing device. For example, processors 1202 may be configured to receive instructions and/or data from storage devices 1212 to execute the functionality of the modules shown in FIG. 12, among others. The computing device 1200 may store data/information to storage devices. It is understood that operations may not fall exactly within any particular modules such that one module may assist with the functionality of another.

Computer program code for carrying out operations may be written in any combination of one or more programming languages, e.g., an object oriented programming language such as Java, Smalltalk, C++ or the like, or a conventional procedural programming language, such as the “C” programming language or similar programming languages.

A computing device 1200 may generate output for display on a screen of gesture-based I/O device or in some examples, for display by a projector, monitor or other display device. It will be understood that gesture-based I/O device 1206 may be configured using a variety of technologies (e.g. in relation to input capabilities: resistive touchscreen, a surface acoustic wave touchscreen, a capacitive touchscreen, a projective capacitance touchscreen, a pressure-sensitive screen, an acoustic pulse recognition touchscreen, or another presence-sensitive screen technology; and in relation to output capabilities: a liquid crystal display (LCD), light emitting diode (LED) display, organic light-emitting diode (OLED) display, dot matrix display, e-ink, or similar monochrome or color display).

In the examples described herein, a gesture-based I/O device 1206 includes a touchscreen device capable of receiving as input tactile interaction or gestures from a user interacting with the touchscreen. Such gestures may include tap gestures, dragging or swiping gestures, flicking gestures, pausing gestures (e.g. where a user touches a same location of the screen for at least a threshold period of time) where the user touches or points to one or more locations of gesture-based I/O device. Gesture-based I/O device and may also include non-tap gestures. Gesture-based I/O device may output or display information, such as graphical user interface, to a user. The gesture-based I/O device may present various applications, functions and capabilities of the computing device including, for example, application to acquire images, view images, process the images and display new images, messaging applications, telephone communications, contact and calendar applications, Web browsing applications, game applications, e-book applications and financial, payment and other applications or functions among others.

Although the present disclosure illustrates and discusses a gesture-based I/O device 1206 primarily in the form of a display screen device with I/O capabilities (e.g. touchscreen), other examples of gesture-based I/O devices may be utilized which may detect movement and which may not comprise a screen per se. In such a case, the computing device 1200 includes a display screen or is coupled to a display apparatus to present new images and GUIs of application. A computing device may receive gesture-based input from input devices 1204 such as a track pad/touch pad, one or more cameras, or another presence or gesture sensitive input device, where presence means presence aspects of a user including for example motion of all or part of the user.

In addition to computing device aspects, a person of ordinary skill will understand that computer program product aspects are disclosed, where instructions are stored in a non-transient storage device (e.g. a memory, CD-ROM, DVD-ROM, disc, etc.) to configure a computing device to perform any of the method aspects stored herein.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise.

Features, integers characteristics, compounds, chemical moieties or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

1.-38. (canceled)

39. A computing device comprising a processor and as storage device coupled thereto, the storage device storing instructions, which when executed by the processor, configure the computing device to evaluate planning data for a plan for a medical procedure by:
providing a prognosis expert system comprising a model configured to generate a patient prognosis from the planning data, wherein the patient prognosis comprises patient outcome data with which to evaluate the planning data; and
processing a respective instance of planning data to provide a respective patient prognosis with which to evaluate the respective instance of the planning data.

40. The computing device of claim 39, wherein the instructions configure the computing device to process two or more respective instances of the planning data to provide respective patient prognoses with which to evaluate the respective instances of the planning data and optimise the planning data; and wherein the respective instances of the planning data comprise data items that are different.

41. The computing device of claim 40, wherein the instructions configure the computing device to automatically generate the respective instances of the planning data using data perturbations generated in response to at least one of upper and lower change bounds and data perturbation rules defined to guide data perturbation.

42. The computing device of claim 39, wherein the model was trained using:
respective planning data from respective expert consultations in respect of respective patients; and
respective post-procedure patient data for the respective patients.

43. The computing device of claim 42, wherein the post-procedure patient data comprises any of:
patient outcome data comprising patient satisfaction, incidents of dislocation, revisions, readmissions, infections, bone fractures;
patient measurement data;
plan execution data; and
post-procedure treatment data.

44. The computing device of claim 39, wherein the model of the prognosis expert system comprises one or more of:
a linear regression model to generate a single binary prognosis score;
a back-propagation neural network to generate a score for each of a plurality of outcomes; and
multiple machine learning models employed sequentially to predict the patient prognosis.

45. The computing device of claim 39, wherein the planning data to be evaluated is generated by an consultation expert system configured to process patient data for a patient to provide an expert consultation comprising the planning data.

46. The computing device of claim 45, wherein either or both of the prognosis expert system and the consultation expert system are trained using cost data for plans defined by respective planning data and wherein the prognosis expert system and the consultation expert system are used or configured to generate the planning data to optimize the patient prognosis while minimizing a plan cost.

47. The computing device of claim 39, wherein:
the patient data comprises: demographic data comprising patient physical or related data comprising one or more of age; weight; height; BMI; gender; ethnicity; patient anatomy particulars comprising pre-operative anatomical measurements and existing implant data; medical history; treated or untreated pathologies; future treatment/proposed procedures; risk factors and comorbidities; and lifestyle/activities of daily living; and
the planning data comprises medical procedure data comprising one or more of a type of the medical procedure and an approach/philosophy to type; a target for the medical procedure selected from an anatomical measurement, an implant measurement and another measurement that is sought to be achieved through the procedure; an additional care recommendation comprising one or more of a pre-procedure therapy/activity, a post-procedure therapy/activity and an additional procedure; and an alternative care recommendation comprising an alternative procedure and/or an alternative therapy.

48. The computing device of claim 39, wherein the medical procedure is an orthopedic surgery procedure.

49. A computing device comprising a processor and as storage device coupled thereto, the storage device storing instructions, which when executed by the processor, configure the computing device to provide a patient prognosis in respect of a plan for an orthopedic medical procedure proposed for a patient by:
receiving a request from a requestor for the patient prognosis, the request comprising planning data for the plan;
processing the request using an expert system comprising a model configured to generate the patient prognosis from the planning data, wherein the patient prognosis comprises patient outcome data with which to evaluate the planning data; and
communicating the patient prognosis to the requestor.

50. The computing device of claim 49, wherein:
the request further comprises patient data, the patient data comprising demographic data comprising patient physical or related data comprising one or more of age; weight; height; BMI; gender; ethnicity; patient anatomy particulars comprising pre-operative anatomical measurements and existing implant data; medical history; treated or untreated pathologies; future treatment/proposed procedures; risk factors and comorbidities; and lifestyle/activities of daily living; and
the planning data comprises medical procedure data comprising one or more of:
a. a type of the medical procedure and an approach/philosophy to type;
b. a target for the medical procedure selected from an anatomical measurement, an implant measurement and another measurement that is sought to be achieved through the procedure;
c. an additional care recommendation comprising one or more of a pre-procedure therapy/activity, a post-procedure therapy/activity and an additional procedure; and
d. an alternative care recommendation comprising an alternative procedure and/or an alternative therapy.

51. The computing device of claim 49, wherein the model of the expert system comprises one or more of:
a linear regression model to generate a single binary prognosis score;
a back-propagation neural network to generate a score for each of a plurality of outcomes; and
multiple machine learning models employed sequentially to predict the patient prognosis.

52. The computing device of claim 49, wherein the expert system comprises a linear regression model to predict a binary prognosis for a specific outcome and a neural network model to predict a timeline associated with the specific outcome.

53. A computing device comprising a processor and as storage device coupled thereto, the storage device storing instructions, which when executed by the processor, configure the computing device to provide an expert consultation in respect of a plan for an orthopedic medical procedure proposed for a patient by:
receiving a request from a requestor for the expert consultation, the request comprising patient data in respect of the patient;
processing the request using an expert system configured to generate the expert consultation from the patient data, wherein the expert consultation comprises planning data to execute the medical procedure; and
communicating the expert consultation to the requestor.

54. The computing device of claim 53, wherein the expert consultation comprises a go/no go recommendation in respect of the performance of the medical procedure.

55. The computing device of claim 53, wherein:
patient data comprises demographic data comprising patient physical or related data comprising one or more of age; weight; height; BMI; gender; ethnicity; patient anatomy particulars comprising pre-operative anatomical measurements and existing implant data; medical history; treated or untreated pathologies; future treatment/proposed procedures; risk factors and comorbidities; and lifestyle/activities of daily living.

56. The computing device of claim 53, wherein:
planning data comprises medical procedure data comprising one or more of a type of the medical procedure and an approach/philosophy to type; a target for the medical procedure selected from an anatomical measurement, an implant measurement and another measurement that is sought to be achieved through the procedure; an additional care recommendation comprising one or more of a pre-procedure therapy/activity, a post-procedure therapy/activity and an additional procedure; and an alternative care recommendation comprising an alternative procedure and/or an alternative therapy.

57. The computing device of claim 56, wherein patient data and planning data each comprise one or more of text data and patient image data of patient anatomy.

58. The computing device of claim 53, wherein the patient data comprises at least one patient image and the expert system comprises a deep neural network to process the at least one patient image and generate at least one target as a portion of the planning data, wherein the at least one target comprises an anatomical measurement, implant measurement or other measurement that is sought to be achieved through the medical procedure.
